(19) United States
(12) Patent Application Publication
(10) Pub. No.: US 2008/0140159 A1
(43) Pub. Date: Jun. 12, 2008

(54) Implantable device for monitoring biological signals

(75) Inventors: Reid Bornhoft, Lino Lakes, MN (US); Gregory J. Loxtercamp, Edina, MN (US); John Arnold, Minneapolis, MN (US); Kathy L. Sherwood, North Oaks, MN (US); Keith Jasperson, Andover, MN (US)

Correspondence Address: FISH & RICHARDSON P.C., P.O. BOX 1022, MINNEAPOLIS, MN, 55440-1022, US

(73) Assignee: Transoma Medical, Inc.

(21) Appl. No.: 11/634,832

(22) Filed: Dec. 6, 2006

(51) Int. Cl. A61N 1/08

(52) U.S. Cl. 607/60

(57) ABSTRACT

An implantable device to be implanted into a body of a patient includes a monitoring system configured to monitor biological signals of the patient, and generate corresponding waveform data. The implantable device includes a first buffer for continually storing the waveform data, a second buffer, and a controller configured to copy waveform data from the first buffer to the second buffer at regular intervals.

### BACKGROUND

Implantable devices are capable of sensing and recording various biological signals from the body, such as, for example, electrocardiogram (ECG) signals. Internal implantable devices offer advantages over external sensing devices that have at least one electrode attached externally to the patient. For example, internal implantable devices can provide a high degree of measurement sensitivity as they decrease the distance between the source of the signals and the sensing device. These highly sensitive measurements are recordable in the electronic components of the implantable device.

In some cases, telemetry is employed to transmit the measurements recorded by the implanted device to an external communication link that processes the data for subsequent analysis and diagnosis. In order to expand the diagnostic capabilities of the implantable device in some cases, there is a need to sense, record, and transmit significant volumes of information. Furthermore, in order to extend the life cycle of the implantable device, the implantable device should be as compact and should consume as little power as possible.

For these and other reasons, there is a need for the present invention.

### SUMMARY

One form of the present invention provides an implantable device to be implanted into a body of a patient. The implantable device includes a monitoring system configured to monitor biological signals of the patient, and generate corresponding waveform data. The implantable device includes a first buffer for continually storing the waveform data, a second buffer, and a controller configured to copy waveform data from the first buffer to the second buffer at regular intervals.

### BRIEF DESCRIPTION OF THE DRAWINGS

The accompanying drawings are included to provide a further understanding of the present invention and are incorporated in and constitute a part of this specification. The drawings illustrate example embodiments and together with the description serve to explain principles of the invention. Other embodiments and many of the intended advantages of the embodiments will be readily appreciated as they become better understood by reference to the following detailed description. The elements of the drawings are not necessarily to scale relative to each other. Like reference numerals designate corresponding similar parts.

FIG. 1 illustrates a block diagram of a telemetry system including an implantable device in accordance with one embodiment.

FIG. 2 illustrates a block diagram of an implantable device in accordance with one embodiment.

FIG. 3 illustrates a block diagram of an application specific integrated circuit for an implantable device in accordance with one embodiment.

FIG. 4 is a block diagram illustrating additional detail of the digital timing and control circuit and memory device shown in FIG. 3 in accordance with one embodiment.

FIG. 5 is a block diagram illustrating additional detail of the digital event detection circuit shown in FIG. 3 in accordance with one embodiment.

FIG. 6A is a graph representative of a human surface ECG signal.

FIG. 6B is a graph illustrating in greater detail a portion of an ECG signal and illustrating generally the typical features of an ECG waveform.

FIG. 7 is a flow diagram illustrating a method for detecting R-waves in an ECG signal in accordance with one embodiment.

FIG. 8 is a diagram illustrating a graph of the magnitude of an example filtered signal provided to the detection circuit shown in FIG. 5 in accordance with one embodiment.

FIG. 9 is a flow diagram illustrating a method of asymptomatic event detection in accordance with one embodiment of the present invention.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

Embodiments relate to an implantable device configured with a signal monitoring system that is configured to continuously measure and selectively record biological signals, such as heart signals, and transmit the recorded biological signal information for subsequent analysis.

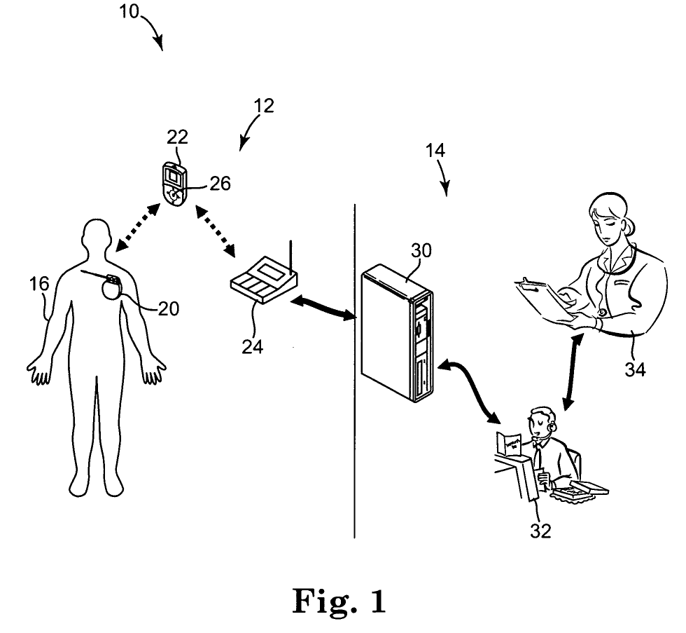

FIG. 1 illustrates telemetry system 10 in accordance with one embodiment. Telemetry system 10 includes patient hub 12 and service hub 14. Patient hub 12 includes a patient 16 provided with an implantable monitoring device (IMD) 20 that is configured to collect biological data from patient 16, a programmable external activator 22 configured to electronically communicate with (i.e., receive and transmit data from) IMD 20, and a base station 24 in telemetric communication with activator 22 that is configured to remotely transmit data collected by IMD 20 and activator 22. In one embodiment, IMD 20 and activator 22 are “paired” when manufactured by programming a unique IMD 20 identification number into a memory bank of activator 22. In this manner, activator 22 is configured to recognize and communicate with a single, specific, and identifiable IMD 20.

Service hub 14 includes service system 30 configured to remotely receive the data collected by IMD 20 that is uploaded to activator 22, and a service technician 32 and a physician or other medical personnel 34 that are enabled to access the data collected by IMD 20 after the data is transmitted to service system 30. Base station 24 of patient hub 12 is linked via a phone system or other communication system to service system 30 of service hub 14, for example by a land-based telephone line system, a wireless communication system, or the Internet, and service technician 32/medical personnel 34 have access to service system 30.

In one embodiment, IMD 20 is a surgically implanted electrocardiogram (ECG) monitoring device configured to continuously capture and selectively record both symptomatic (i.e., patient detected) and asymptomatic (i.e., non-patient detected, or IMD 20 detected) ECG events. In one embodiment, IMD 20 is configured to capture and record trending ECG waveform data based on periodic timed triggering of IMD 20. In this regard, ECG events and other biological signals are monitored and recorded within IMD 20, which is configured with transceiver capabilities for uploading data to activator 22. In one embodiment, activator 22 uploads the biological data from the patient 16 and is configured to wirelessly transmit the data to the base station 24 when the patient 16 is, for example, within wireless fidelity (WiFi) range of base station 24.

In one embodiment, activator 22 is rechargeable and sized to be worn externally or carried by patient 16. In one embodiment, activator 22 is a computational device including memory and programmable software that combine to enable activator 22 to program IMD 20, display waveforms of data collected by IMD 20 on a real-time basis, respond to patient commands storing symptomatic data collected by IMD 20, store asymptomatic data collected by IMD 20, upload data from IMD 20, download data to IMD 20, and transmit data to service system 30 via base station 24.

One embodiment of activator 22 includes a patient interface 26 that is configured to enable patient 16 to send an activation signal to selectively activate IMD 20 to record a symptomatic ECG event (e.g., an anomalous cardiac event detected by patient 16) and, in one form of this embodiment, upload information from IMD 20 to activator 22, and then to service system 30 via base station 24 during the event. In one embodiment, activator 22 passively uploads ECG events recorded by IMD 20 at regular time intervals (e.g., daily) and transmits this data to service system 30 via base station 24. In one embodiment, activator 22 is configured to receive information, such as, for example, clock synchronization information transmitted from service system 30 through base station 24, for activator 22 and/or for downloading to IMD 20.

Base station 24 may be coupled to service system 30 in a variety of suitable ways. For example, base station 24 and service system 30 may be coupled by telephone lines, wireless communication, or the Internet. Other suitable communication links between base station 24 and service system 30 are also acceptable. Regardless of the communication link between base station 24 and service system 30, technician 32 has access to the patient 16 data measured by IMD 20.

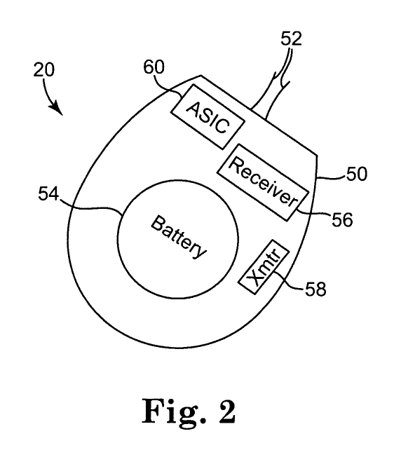

FIG. 2 illustrates a block diagram of IMD 20 in accordance with one embodiment. In one embodiment, IMD 20 includes a case 50, leads 52, a battery 54, a receiver 56, a transmitter 58, and an application specific integrated circuit (ASIC) 60 contained within case 50. In one embodiment, case 50 is a sealed titanium case sized to house various components of IMD 20, such as battery 54, receiver 56, transmitter 58, and ASIC 60. When implanted, IMD 20 can measure biological signals, such as ECG potentials, across leads 52 and store segments of the biological signal waveforms within ASIC 60. In one embodiment, one of leads 52 is coupled to an extending lead having a remote tip electrode, and the other of leads 52 is coupled to case 50, such that an ECG potential is measurable between the remote tip electrode and case 50. In one embodiment, ASIC 60 is coupled to an IMD memory device, such as a static random access memory (SRAM), which can be configured to store segments of the signal waveforms for subsequent transmission to activator 22.

Receiver 56 is configured to receive commands signals, for example, from activator 22. In one embodiment, activator 22 sends an activation signal that indicates that a segment of an ECG waveform should be recorded and then transmitted. When such an activation signal is received, receiver 56 can be configured to pass the activation signal to ASIC 60 so that segments of the ECG waveform are recorded. In some embodiments, the waveform signals can be stored in ASIC 60 and/or an IMD memory device external from ASIC 60. The recorded segments of the ECG waveform can then be sent to transmitter 58 for transmission to activator 22. In some embodiments, the signals can be transmitted directly to activator 22 rather than first storing them in ASIC 60 and/or an IMD memory device.

Once measured and transmitted, the data is available to service system 30 via the link between base station 24 and system 30. Thereafter, technician 32/medical personnel 34 have access to the measured signals. As such, the information in the measured signals may be used by a physician to remotely diagnose a condition of patient 16, to observe and record the measured signals, and/or to further instruct IMD 20 based on the measured signals.

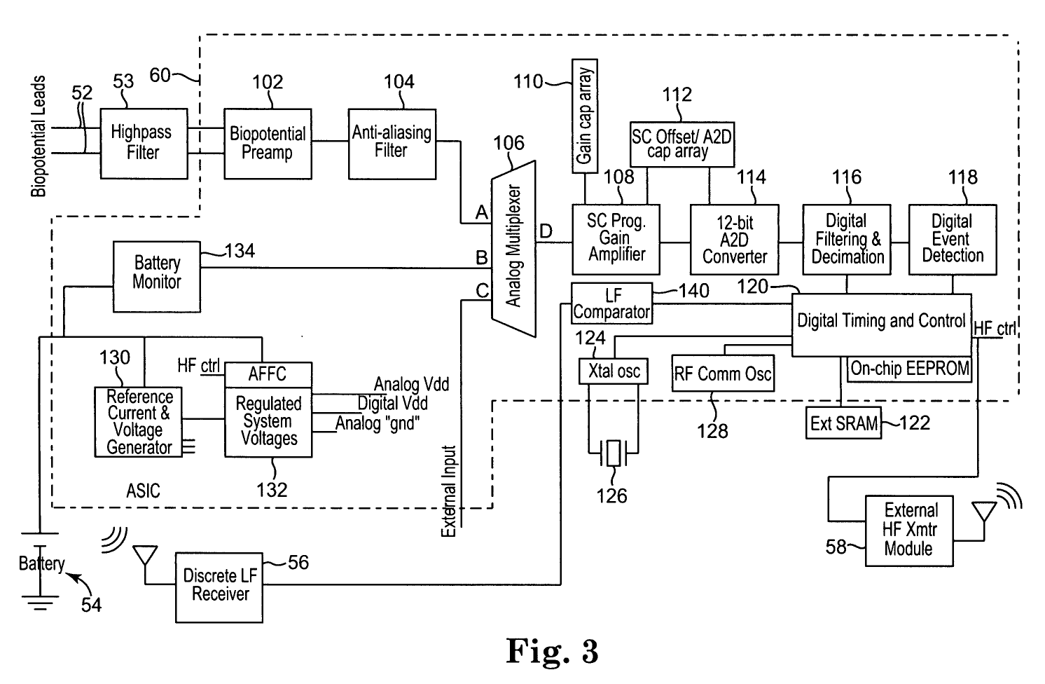

FIG. 3 illustrates a block diagram of one embodiment of an ASIC 60 (represented with a dashed-line). Biopotential leads 52 are coupled to ASIC 60 via a high-pass filter network 53. Battery 54, receiver 56, and transmitter 58 are electrically coupled to ASIC 60. In operation, biological signals, such as ECG events, are captured between biopotential leads 52 and transmitted into ASIC 60 via the high-pass filter 53.

Once biological signals enter ASIC 60, they are received by biopotential preamplifier 102. Since the received biological signals can be relatively weak, biopotential preamplifier 102 in one embodiment is configured to boost the strength of the biological signals. While biopotential preamplifier 102 is configured to boost the biological signals, it is also configured to minimize the amount of random noise added to the weak signals by preamplifier 102 itself.

Anti-aliasing filter 104 receives the boosted signals from biopotential preamplifier 102. In one embodiment, anti-aliasing filter 104 is configured to restrict the bandwidth of the signals to prepare it for digital sampling. In one embodiment, anti-aliasing filter 104 is a three-pole low pass active filter circuit, which provides an appropriate cut off frequency for the biological signals for later sampling.

The filtered signals from anti-aliasing filter 104 are passed to analog multiplexer 106. Analog multiplexer 106 is configured to receive a plurality of analog signals at one of its input terminals A, B, and C. In one embodiment, the biological signals from anti-aliasing filter 104 are received at input terminal A of analog multiplexer 106. The other two input terminals B and C are configured to receive other signals that can be selected by analog multiplexer 106, as will be discussed in more detail below.

When selected by analog multiplexer 106, the biological signals transmitted through biopotential preamplifier 102 and anti-aliasing filter 104 can be sent to switched capacitor programmable gain amplifier 108 (hereafter programmable gain amplifier 108) via output terminal D of analog multiplexer 106. Programmable gain amplifier 108 is configured to appropriately scale the biological signals in order to appropriately prepare the signals for analog to digital conversion. In one embodiment, programmable gain amplifier 108 allows a programmable gain to be applied to the output such that a very accurate gain at discrete time intervals is obtained for very low current. In one embodiment, a gain capacitor array 110 (hereafter capacitor array 110) and a shared switched capacitor offset and analog-to-digital converter array 112 (hereafter shared capacitor array 112) are employed in conjunction with programmable gain amplifier 108 in order to scale the signals.

After the signals have been appropriately scaled, analog-to-digital converter 114 samples the signals in order to convert analog signals to digital signals. In one embodiment, analog-to-digital converter 114 is a 12-bit charge redistribution converter. In one embodiment, analog-to-digital converter 114 shares shared capacitor array 112 with programmable gain amplifier 108.

The converted digital signal is submitted to digital filtering circuit 116. In one embodiment, digital filtering circuit 116 filters the sampled data at a programmed sample rate. The filtered data is down sampled and stored in a buffer memory. Digital event detection circuit 118 is utilized in some embodiments to analyze the sampled data in order to recover and detect particular events in the signal. For example, digital event detection circuit 118 may detect when R-waves of the QRS complexes occur in an ECG signal.

Digital timing and control circuit 120 is coupled to digital filtering circuit 116 and digital event detection circuit 118. Digital timing and control circuit 120 coordinates and controls many of the digital operations of ASIC 60. In one embodiment, digital timing and control circuit 120 includes an on-chip EEPROM, which may be externally programmable. In one embodiment, an additional register within Digital Timing and Control 120 is employed by external software to further increase the gain accuracy by providing a calibration value to apply to the data once received. Also in one embodiment, an external memory device 122 is provided and coupled to ASIC 60. In one case, ASIC 60 and memory device 122 may be coupled together in a ball grid array for integration within IMD 20. In one example embodiment, memory device 122 is a static random access memory (SRAM) device.

In one embodiment, crystal oscillator circuit 124 provides timing control and clocking to the various digital and analog circuitry on ASIC 60, including digital timing and control circuit 120. In one case, crystal oscillator circuit 124 includes crystal 126. In one embodiment crystal 126 is provided off ASIC 60 and the remaining portions of crystal oscillator circuit 124 are integrated within ASIC 60. The clock signal generated by crystal oscillator circuit 124 is provided to digital timing and control circuit 120 so that it controls digital operations within ASIC 60 in accordance with the generated clock. In addition, in one embodiment the clock signal or control signals derived from the clock are utilized by analog multiplexer 106, programmable gain amplifier 108, and analog-to-digital converter 114.

In one embodiment, a radio-frequency communication oscillator 128 provides clocking to the various digital circuitries on ASIC 60. In one embodiment, receiver 56 is an external receiver composed of discrete parts and transmitter module 58 is an external high-frequency transmitter module. The clock signal generated by radio-frequency communication oscillator 128 is programmable to provide for variable bit rates for transmissions, and in one embodiment controls high-frequency output to transmitter 58.

Power is provided to ASIC 60 via battery 54, which in one embodiment is external to ASIC 60. Battery 54 is coupled to reference current and voltage generator 130, regulator circuit 132, and battery monitor 134. In one embodiment, battery monitor 134 periodically, or on demand, monitors battery 54 in order to determine its approximate remaining life cycle. In one case, this information can be transferred, via input B of analog multiplexer 106, and selected for transmission to digital circuitry and later transmission outside ASIC 60. In this way, the approximate remaining life of battery 54 can be determined external to IMD 20 and remotely therefrom.

Reference current and voltage generator 130 is coupled to battery 54 and generates various bias currents for use by the various components of ASIC 60. Regulator circuit 132 also is coupled to battery 54 and generates a plurality of voltages that are also used by various components of ASIC 60. For example, regulator circuit 132 can generate an analog source voltage V_{dd(analog)}, a digital source voltage V_{dd(digital),} and an analog ground voltage Vₐₙₐ. These various voltages are also provided to various functional blocks within ASIC 60.

ASIC 60 is also coupled to both receiver 56 and transmitter 58, each of which are external to ASIC 60 in one embodiment. In one embodiment, receiver 56 is coupled to a low frequency comparator circuit 140, which in turn is coupled to digital timing and control circuit 120. As such, in one embodiment information transmitted remotely to IMD 20 can be received in receiver 56 and communicated to digital timing and control 120 for further processing and control. In one embodiment, external transmitter module 58 is coupled to a high-frequency control terminal of digital timing and control circuit 120. As such, digital timing and control circuit 120 can control the transmission of data to the transmitter module 58 for transmission outside IMD 20.

In one embodiment, IMD 20 and activator 22 are configured to communicate with each other using wireless radio frequency (RF) communications. In one form of the invention, the relationship between activator 22 and IMD 20 is a master-slave relationship, with activator 22 being the master, and IMD 20 being the slave. In one embodiment, all communications between IMD 20 and activator 22 are initiated by the activator 22.

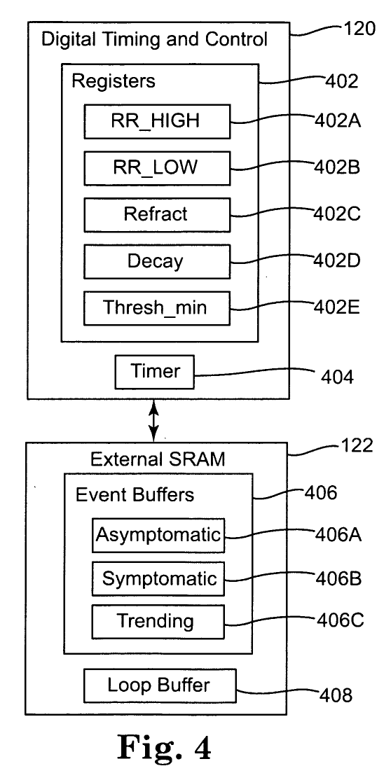

FIG. 4 is a block diagram illustrating additional detail of the digital timing and control circuit 120 and memory device 122 shown in FIG. 3 in accordance with one embodiment. In the illustrated embodiment, digital timing and control circuit 120 includes a plurality of registers 402A-402E (collectively referred to as registers 402), and a timer 404. In one embodiment, the registers 402 are programmable via the activator 22. RR_HIGH register 402A stores a high rate R-R interval value that defines a high rate event threshold for detecting a high rate or tachycardia rhythm event. RR_LOW register 402B stores a low rate R-R interval value that defines a low rate event threshold for detecting a low rate or bradycardia rhythm event. In one embodiment, RR_HIGH register 402A and RR_LOW register 402B are implemented in a single configuration register. Refract register 402C stores a refract parameter that defines a refractory period following the detection of an R-wave. Detection of R-waves is blanked or suspended during the refractory period, which avoids possible double counting of a wide QRS complex and also avoids noise sources.

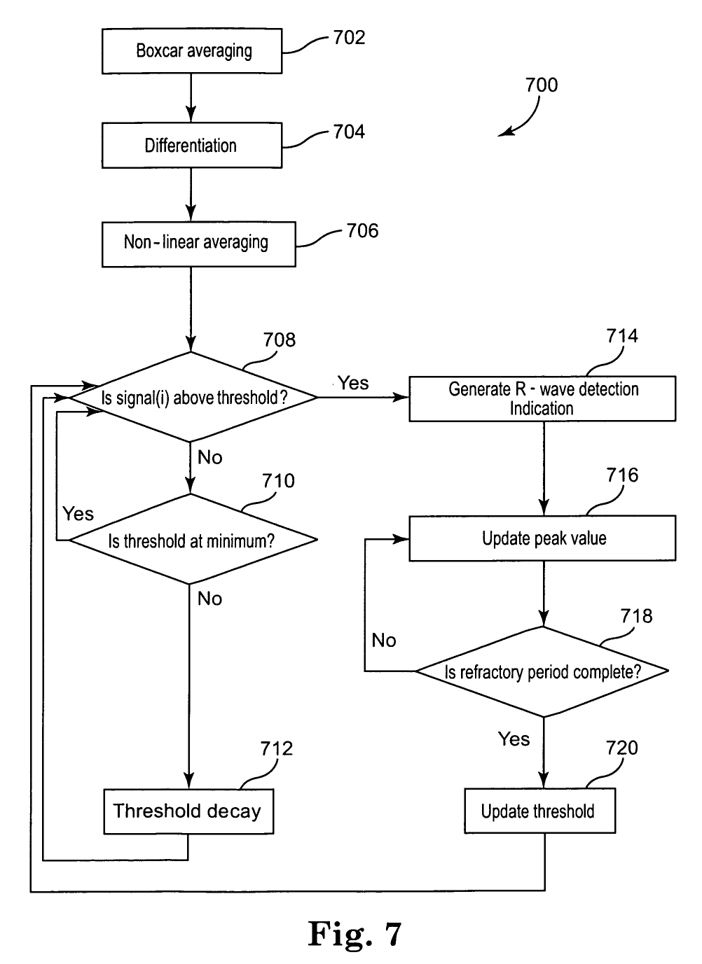

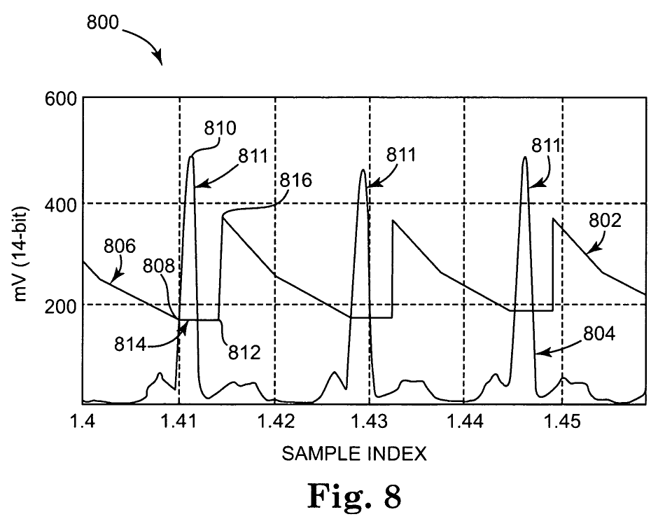

Detection of R-waves according to one embodiment is accomplished using an adaptable or variable detection threshold that is continually adjusted based on a decay parameter and the magnitude of detected R-waves. Decay register 402D stores the decay parameter, which is a value that determines the rate of decay of the detection threshold following the end of the refractory period. The detection threshold value In one embodiment, the registers 402 are programmable via the activator 22 decays down in order to detect the next R-wave. Thresh_min register 402E stores a minimum threshold parameter. The threshold decays down until then next R-wave is detected, or until it reaches the value programmed into the thresh_min register 402E. Once this minimum threshold value is reached, the detection threshold remains constant at this minimum value and does not decay any further until after an R-wave is detected. This minimum threshold value prevents the detection threshold from going too low and detecting noise. The detection of R-waves using a variable detection threshold according to one embodiment is described in further detail below with reference to FIGS. 7 and 8.

As shown in FIG. 4, memory device 122 includes a plurality of event buffers 406A-406C (collectively referred to as buffers 406) and a loop buffer 408. IMD 20 continually samples and stores data in loop buffer 408 at a down-sampled rate (e.g., 200 Hz). The loop buffer 408 stores the sampled and filtered data in a continuous first-in first-out basis. Data from the loop buffer 408 is copied into the appropriate buffers 406 by digital timing and control circuit 120 upon the occurrence of certain events, and is later transmitted from buffers 406 to activator 22. In one embodiment, memory device 122 is a 1 MB SRAM device arranged in a 512K by 16 bit manner.

In one embodiment, buffers 406 store ECG data events. Buffer 406A is an asymptomatic buffer that contains ECG waveform data based on events that are automatically detected by IMD 20. Buffer 406B is a symptomatic buffer that contains ECG waveform data based on patient initiated event triggering via patient interface 26 of activator 22. Buffer 406C is a trending buffer that contains ECG waveform data based on periodic timed triggering by IMD 20.

In one embodiment, each of the buffers 406 and 408 is managed as a first-in first-out (FIFO) buffer, where data is stored sequentially and wraps to the beginning of the buffer when the end of the buffer is reached, but protected events are not overwritten. If a buffer 406 fills up prior to being cleared by the activator 22, stored event data are overwritten with the exception of protected events, in which case the location following the protected space is overwritten. If a buffer 406 is full of protected events, no overwriting occurs in that buffer 406 in one embodiment.

In one embodiment, loop buffer 408 includes 7.3 minutes of data space, and buffers 406 include a total of 43 minutes of data space. In one embodiment, loop buffer 408 stores a total of 87,381 samples of data, or 624 segments of data (with 140 samples per segment).

In one embodiment, asymptomatic buffer 406A includes 28.0 minutes of data space. When IMD 20 makes an asymptomatic event detection, thirty seconds preceding the detection and thirty seconds following the detection are copied from loop buffer 408 to asymptomatic buffer 406A. Thus, asymptomatic buffer 406A can store twenty-eight asymptomatic events (with each event being one minute in duration).

In one embodiment, IMD 20 calculates an R-R interval (i.e., time interval between consecutive R-waves) on a beat-by-beat basis, and automatically copies a portion of the ECG waveform from loop buffer 408 to asymptomatic buffer 406A when the R-R interval is outside of programmed limits (i.e., indicating the occurrence of an asymptomatic event). High and low limits are programmed into IMD 20 via activator 22 and stored in RR_HIGH register 402A and RR_LOW register 402B, respectively. The periods between the R-wave detection points are compared to the RR_HIGH and RR_LOW parameters stored in the registers 402A and 402B in order to determine if an asymptomatic event occurred.

Within the asymptomatic class of events, there are two types—high rate and low rate. IMD 20 detects and stores a high rate asymptomatic event when N of M consecutive R-R intervals are each less than the value stored in register 402A, where N and M are integers, and N is greater than or equal to M/2. The values for N and M are small enough in one embodiment to provide a relatively fast detection window for detecting tachycardia rhythm events (which can be short duration events), but large enough to help provide noise immunity (i.e., very small values for N and M could result in noise causing an erroneous detection of a high rate event). In one embodiment, M is in the range of 5 to 11, and N is in the range of 3 to 9. In one embodiment, M equals 8 and N equals 6. In this embodiment, IMD 20 detects and stores a high rate asymptomatic event when 6 of 8 successive R-R intervals are less than (i.e., faster than) the interval programmed into the RR_HIGH register 402A. IMD 20 detects and stores a low rate asymptomatic event in one embodiment when a single R-R interval exceeds (i.e., is slower than) the interval programmed into the RR_LOW register 402B (i.e, a single beat-to-beat rate that is slower than the preset limit).

Within the high rate asymptomatic event type, a sub-type of protected high rate asymptomatic event exists in one embodiment, which occurs when the heart rate exceeds a threshold number of beats per minute during the detected event. In one embodiment, the threshold number of beats per minute is 220 beats per minute (bpm). A high rate event is automatically marked as a protected event by digital timing and control circuit 120 if one of the eight consecutive R-R intervals that triggered the event is less than the RR_HIGH number of sample periods, which corresponds to a 220 bpm heart rate.

In one embodiment, the storage space in asymptomatic buffer 406A is automatically overwritten on a first-in first-out basis when twenty-eight minutes of storage is exceeded, except for event data taken when the heart rate exceeds 220 bpm (i.e., a protected event). Data taken when the heart rate exceeds 220 bpm is written into the asymptomatic buffer 406A with overwrite protection, and is not overwritten until it has been uploaded to the activator 22. In one embodiment, asymptomatic buffer 406A stores a total of 12,000 samples of data, or 86 segments of data (with 140 samples per segment). The last segment in the buffer 406A will have fewer than 140 samples (i.e., 100 samples rather than 140 samples).

In one embodiment, symptomatic buffer 406B includes 14.0 minutes of data space. When a symptomatic detection is triggered by a patient using patient interface 26 of activator 22, five minutes preceding the detection and two minutes following the detection are written from loop buffer 408 to symptomatic buffer 406B. Thus, symptomatic buffer 406B according to one embodiment can store two symptomatic events, with each event being seven minutes in duration. For example, a patient may be feeling symptoms and presses a button on the patient interface 26 of the activator 22, which sends a corresponding command to IMD 20 to capture a symptomatic event. In one embodiment, the storage space in symptomatic buffer 406B is automatically overwritten on a first-in first-out basis when fourteen minutes of storage is exceeded. In one embodiment, symptomatic buffer 406B stores a total of 84,000 samples of data, or 600 segments of data (with 140 samples per segment).

In one embodiment, trending buffer 406C includes 1.0 minute of data space. Digital timing and control circuit 120 according to one embodiment writes ten seconds of data from loop buffer 408 to trending buffer 406C every four hours. Thus, a total of one minute (six trending events, each having a duration of ten seconds) of trending data is written to trending buffer 406C every twenty-four hours. In one embodiment, an internal timer 404 within digital timing and control circuit 120 initiates trending events automatically at regular intervals. In one embodiment, the storage space in trending buffer 406C is automatically overwritten on a first-in first-out basis when one minute of storage is exceeded. In one embodiment, trending buffer 406C stores a total of 2,000 samples of data, or 15 segments of data (with 140 samples per segment). The last segment in the buffer 406C will have fewer than 140 samples (i.e., 40 samples rather than 140 samples).

In one embodiment, each full segment (i.e., 140 samples) of data stored in the buffers 406 is thirty-six bytes in size, and each sample within a segment is 12-bits in size. Thus, a 36-byte segment will contain twenty-four samples. In one embodiment, the data stored in the buffers 406 for each event includes EGC waveform data, an event type indicator (i.e., symptomatic, asymptomatic, or trending) and a time stamp of the waveform data.

The activator 22 generates various commands, including commands to read and write to the registers 402 of the IMD 20, and request data from the buffers 406 of the IMD 20, and transmits the commands to IMD 20. IMD 20 responds to the commands received from the activator 22, and transmits requested data to activator 22. In one embodiment, data in the buffers 406 is uploaded to the activator 22 at regular intervals (e.g., at least once per day), and buffers 406 are cleared after the data is uploaded. In one embodiment, if a patient has not initiated an upload command within a 20-hour period using activator 22, the activator 22 automatically signals the IMD 20 to upload the contents of the buffers 406 to the activator 22. Twenty hours from the last successful data upload (either patient-activated or non-patient activated), an upload request command is generated by the activator 22 and transmitted to the IMD 20. Upload request commands are generated every 30 minutes thereafter up to and including a point 24 hours after the last successful upload until a successful data upload is achieved. In one form of the invention, the activator 22 includes a sufficiently large non-volatile data buffer to allow for storage of 630 minutes of waveform data from buffers 406 along with associated time stamps and type of event information, which corresponds to fourteen or more days of information from IMD 20.

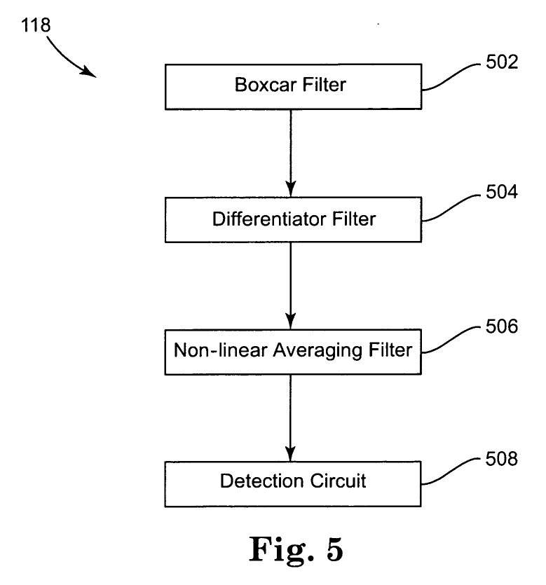

In one embodiment, asymptomatic events are detected by digital event detection circuit 118 (FIG. 3). FIG. 5 is a block diagram illustrating additional detail of the digital event detection circuit 118 shown in FIG. 3 in accordance with one embodiment. Digital event detection circuit 118 analyzes the sampled and filtered data output by digital filtering circuit 116 and detects asymptomatic events based on the received data. In the illustrated embodiment, digital event detection circuit 118 includes a boxcar filter 502, a differentiator filter 504, a non-linear averaging filter 506, and a detection circuit 508. The boxcar filter 502 receives the sampled and filtered data (e.g., 12-bit data) output by digital filtering circuit 116, and performs on averaging function on the received data. The boxcar filter 502 outputs filtered data to differentiator filter 504, which performs a differentiation function on the received data. Non-linear averaging filter 506 receives filtered data from differentiator filter 504, and performs a non-linear averaging function using the absolute value of the received data. Non-linear averaging filter 506 outputs filtered data to detection circuit 508.

In one embodiment, boxcar filter 502 is a 4-point boxcar [1 1 1 1]/4, differentiator filter 504 is a 5-point differentiator filter [2 1 0 −1 −2]/6, and non-linear averaging filter 506 is a 16-point averaging filter [1 1 1 1 1 1 1 1 1 1 1 1 1 1 1 1]/16. In one embodiment, filters 502, 504, and 506 operate at the storage sample rate (e.g., 200 Hz). Filters 502, 504, and 506 are described in further detail below with reference to FIG. 7.

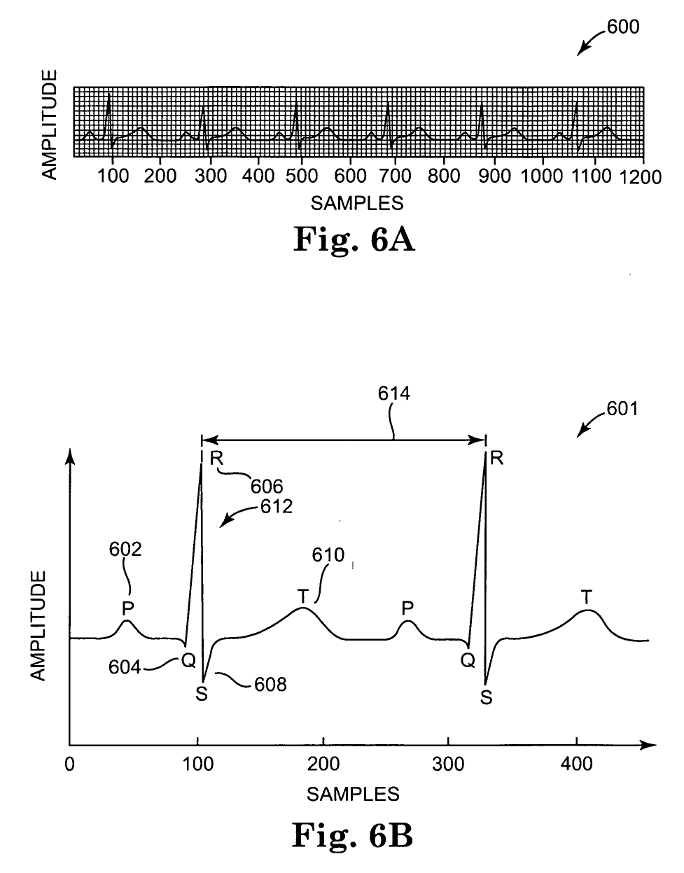

Filters 502, 504, and 506 smooth the signal received from digital filtering circuit 116, and help to eliminate noise from the signal. The filtered data output by the filter 506 and used in the detection by detection circuit 508 is called diction or rate-channel data. Detection circuit 508 receives the rate-channel data from non-linear averaging filter 506, and based on the received data, determines when R-waves of the QRS complexes occur. The operation of event detection circuit 118 is described in further detail below with reference to FIG. 7, following a description of QRS complexes and R-waves, which are shown in FIGS. 6A and 6B.

FIG. 6A is a graph 600 representative of a human surface ECG signal. FIG. 6B is a graph 601 illustrating in greater detail a portion of an ECG signal and illustrating generally the typical features of an ECG waveform. As illustrated, the ECG waveform includes a P-wave 602, a Q-wave 604, an R-wave 606, an S-wave 608, and a T-wave 610. P-wave 602 is caused by excitation of the atria. Together, Q-wave 604, R-wave 606, and S-wave 608 form what is commonly referred to as a QRS-complex, indicated at 612, which results from the excitation (de-polarization) of the ventricles. T-wave 610 results from recovery (re-polarization) of the ventricles. The interval between the peaks of consecutive R-waves is referred to as an R-R interval 614, and corresponds to one cycle of the ECG, sometimes referred to as a cardiac cycle or heart beat.

ECGs are reflective of various aspects of the physical condition of the human heart and are employed, for example, to measure the rate and regularity of heartbeats, to detect the presence of damage to the heart, to monitor the effects of drugs, and for providing operating information to devices used to regulate heartbeats (e.g., defibrillators).

FIG. 7 is a flow diagram illustrating a method 700 for detecting R-waves 606 (FIG. 6) in an ECG signal in accordance with one embodiment. In one embodiment, event detection circuit 118 (FIG. 3) is configured to perform method 700. At 702, boxcar filter 502 (FIG. 5) receives the sampled and filtered data output by digital filtering circuit 116, and performs on averaging function on the received data according to the following Equation I:

$$y = \frac{1}{4} {\sum }_{i = 1}^{4} x ( i ) \qquad Equation I$$

Where:
y=filtered sample output by filter 502;
i=index for identifying samples of data input to filter 502; and
x(i)=the ith sample of data input to filter 502.

At 704, differentiator filter 504 (FIG. 5) receives the filtered data from boxcar filter 502 and performs a differentiation function on the received data according to the following Equation II:

$$y = \frac{1}{6} {\sum }_{i = 1}^{6} ( 2 x ( i - 4 ) + x ( i - 3 ) - x ( i + 1 ) - 2 x ( i ) ) \qquad Equation II$$

Where:
y=filtered sample output by filter 504;
i=index for identifying samples of data input to filter 504; and
x(i)=the ith sample of data input to filter 504.

At 706, non-linear averaging filter 506 (FIG. 5) receives filtered data from differentiator filter 504, and performs a non-linear averaging function using the absolute value of the received data according to the following Equation III:

$$y = \frac{1}{16} {\sum }_{i = 1}^{16} \vert x ( i ) \vert \qquad Equation III$$

Where:
y=filtered sample output by filter 506;
i=index for identifying samples of data input to filter 506; and
x(i)=the ith sample of data input to filter 506.

The output, y, of non-linear averaging filter 506 is also referred to herein as rate-channel samples, signal(i), where “i” is an index for identifying individual samples of rate-channel data. Non-linear averaging filter 506 outputs the rate-channel samples, signal(i), to detection circuit 508. At 708, detection circuit 508 (FIG. 5) receives a rate-channel sample, signal(i), from non-linear averaging filter 506, and determines whether the sample, signal(i), is above the current detection threshold value. If it is determined at 708 that the sample, signal(i), is not above the detection threshold value, the method moves to 710. At 710, detection circuit 508 compares the detection threshold value to a minimum threshold value, and determines whether the detection threshold is at the minimum threshold value. In one embodiment, the minimum threshold value used at 710 is the value programmed into thresh_min register 402E of digital timing and control circuit 120. If it is determined at 710 that the detection threshold value is at the minimum threshold value, the method 700 returns to 708 to process the next sample. If it is determined at 710 that the detection threshold value is not at the minimum threshold value, the method 700 moves to 712.

At 712, detection circuit 508 calculates a new detection threshold value by performing a decay function on the previous detection threshold value. In one embodiment, the new detection threshold value is calculated based on the following Equation IV:

$$newthreshold ( i ) = MAX ( threshold ( i ) - MIN ( ( \frac{threshold ( i )}{32 \times {2}^{( DECAY - 1 )}} ) , 1 ) , thresh\_min ) \qquad Equation IV$$

Where:
newthreshold(i)=newly calculated detection threshold value;
MAX=function that determines which of the parameters in parentheses is the largest;
threshold(i)=previous value of the detection threshold;
MIN=function that determines which of the parameters in parentheses is the smallest;
DECAY=programmable decay value stored in register 402D of circuit 120; and
thresh_min=minimum threshold value programmed into thresh_min register 402E.

After the new detection threshold value is calculated at 712, method 700 returns to 708 to process the next sample.

If it is determined at 708 that the sample, signal(i), is above the detection threshold value, the method 700 moves to 714. At 714, detection circuit 508 generates an R-wave detection indication. At 716, detection circuit 508 updates a peak value for the rate-channel signal, signal(i), according to the following Equation V:

$$peak ( i ) = max ( signal ( i ) , peak ( i - 1 ) ) \qquad Equation V$$

Where:
peak(i)=current value of the local maximum of the rate-channel signal, signal (i);
max=function that determines which of the parameters in parentheses is the largest;
signal(i)=current rate-channel signal sample; and
peak(i−1)=previous value of the local maximum of the rate-channel signal, signal (i).

At 718, detection circuit 508 determines whether the refractory period is complete. In one embodiment, a value identifying the length of the refractory period is stored in refract register 402C of digital timing and control circuit 120. If it is determined at 718 that the refractory period is not complete, the method 700 returns to 716 to update the peak value based on the next signal sample. If it is determined at 718 that the refractory period is complete, the method 700 moves to 720. At 720, detection circuit 508 calculates a new detection threshold value based on the following Equations VI and VII:

$$avg\_peak ( i ) = ( \begin{matrix}( 3 / 4 ) * avg\_peak ( i - 1 ) + \\ ( 1 / 4 ) * current\_peak\end{matrix} ) \qquad Equation VI$$

Where:
avg_peak(i)=current average peak value of the rate-channel signal, signal (i);
avg_peak(i−1)=previous average peak value of the rate-channel signal, signal (i); and
current_peak=current peak value of the rate-channel signal, signal (i), as determined at 716 in method 700.

$$newthreshold ( i ) = ( 3 / 4 ) ( avg\_peak ( i ) ) \qquad Equation VII$$

Where:
newthreshold(i)=newly calculated detection threshold value; and
avg_peak(i)=current average peak value calculated from Equation VI.

After the new detection threshold value is calculated at 720, method 700 returns to 708 to process the next sample. The detection of R-waves and the updating of the detection threshold value are described in further detail below with reference to FIG. 8.

FIG. 8 is a diagram illustrating a graph 800 of the magnitude of an example rate-channel filtered signal (signal(i)) 804 provided to the detection circuit 508 shown in FIG. 5 in accordance with one embodiment. FIG. 8 also shows a graph of the detection threshold value 802 used in the method 700. As indicated at 806 in FIG. 8, the detection threshold value 802 continually decays (due to the updating at 712 of method 700) until the signal 804 rises up above the threshold 802, which is indicated at 808. At point 808 in the signal 804, the detection circuit 508 will determine at 708 in method 700 that the signal is now above the detection threshold, and will make an R-wave detection indication at 714, which indicates that an R-wave 811 has been detected. The signal 804 continues to rise after point 808, and detection circuit 508 continues to update the peak value of the signal at 716 of method 700 during the refractory period 814 (i.e., the time period between points 808 and 812) to determine the value at the peak 810 of the R-wave 811. The detection threshold value 802 remains constant during the refractory period 814, and at the end of the refractory period (indicated at 812), a new detection threshold value is calculated at 720 of method 700, and the graph of the detection threshold 802 jumps up to the new threshold value 816 accordingly. Next, the detection threshold 802 again begins to decay (due to the updating at 712 of method 700), and the process is repeated to detect the next R-wave 811 in the signal 804.

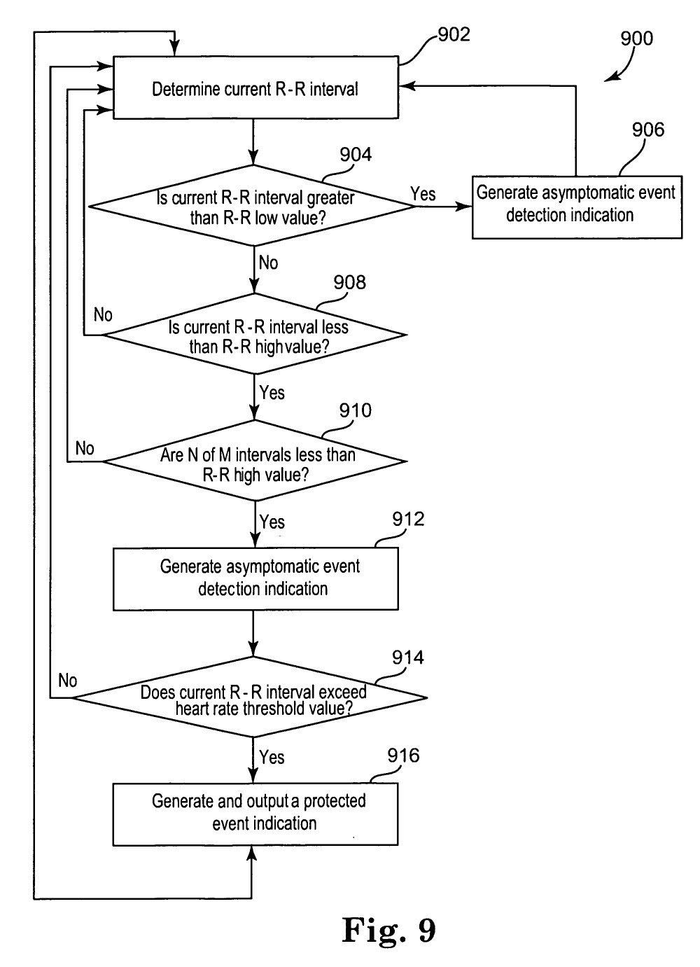

FIG. 9 is a flow diagram illustrating a method 900 of asymptomatic event detection in accordance with one embodiment of the present invention. In one embodiment, digital event detection circuit 118 (FIG. 3) is configured to perform method 900. At 902, detection circuit 508 (FIG. 5) determines the time interval between a currently detected R-wave and a previously detected R-wave immediately preceding the currently detected R-wave. This time interval is referred to as the current R-R interval. At 904, detection circuit 508 compares the current R-R interval to the value in RR_LOW register 402B, and determines if the R-R interval is greater than the R-R low value stored in the register 402B (i.e., slower than the low rate threshold). If it is determined at 904 that the current R-R interval is greater than the R-R low value stored in register 402B, the method 900 moves to 906. At 906, detection circuit 508 generates a low rate asymptomatic event detection indication, outputs the indication to digital timing and control circuit 120, and returns to 902 of method 900.

If it is determined at 904 that the current R-R interval is not greater than the R-R low value stored in register 402B, the method 900 moves to 908. At 908, detection circuit 508 compares the current R-R interval to the value in RR_HIGH register 402A, and determines if the current R-R interval is less than the R-R high value stored in the register 402A (i.e., faster than the high rate threshold). If it is determined at 908 that the current R-R interval is not less than the R-R high value stored in register 402A, the method 900 returns to 902. If it is determined at 908 that the current R-R interval is less than the R-R high value stored in register 402A, the method 900 moves to 910.

At 910, detection circuit 508 examines M successive R-R intervals (i.e., the R-R interval determined at 902 and the M−1 intervals preceding this R-R interval), and determines whether N of the M intervals are less than the R-R high value stored in register 402A, where N and M are integers, and N is greater than or equal to M/2. In one embodiment, M=8 and N=6. If it is determined at 910 that the N of M intervals are not less than the value stored in register 402A, the method 900 returns to 902. If it is determined at 910 that N of M intervals are less than the value stored in register 402A, the method moves to 912. At 912, detection circuit 508 generates a high rate asymptomatic event detection indication, and outputs the indication to digital timing and control circuit 120.

At 914, event detection circuit 508 determines whether the current R-R interval determined at 902 corresponds to a heart rate that exceeds a high rate threshold value. In one embodiment, the threshold value used at 914 is a value corresponding to 220 beats per minute. If it is determined at 914 that the current R-R interval does not correspond to a heart rate that exceeds the threshold value, the method 900 returns to 902. If it is determined at 914 that the current R-R interval corresponds to a heart rate that exceeds the threshold value, the method 900 moves to 916. At 916, event detection circuit 508 generates a protected event indication, and outputs the indication to digital timing and control circuit 120, which causes digital timing and control circuit 120 to designate the currently detected asymptomatic event as a protected event in the buffer 406A. The method 900 then returns to 902.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. An implantable device to be implanted into a body of a patient, the implantable device comprising:
a monitoring system configured to monitor biological signals of the patient, and generate corresponding waveform data;
a first buffer for continually storing the waveform data;
a second buffer; and
a controller configured to copy waveform data from the first buffer to the second buffer at regular intervals.

2. The implantable device of claim 1, and further comprising:
a communication system configured to transmit data from the second buffer to an external device.

3. The implantable device of claim 2, wherein the communication system is configured to transmit data from the second buffer to the external device at regular intervals.

4. The implantable device of claim 2, wherein the communication system is configured to transmit data from the second buffer to the external device at least once per day.

5. The implantable device of claim 1, wherein the communication system is configured to communicate with the external device via wireless radio-frequency communications.

6. The implantable device of claim 1, and further comprising:
a third buffer; and
wherein the controller is configured to copy waveform data from the first buffer to the third buffer upon detection of an event by the implantable device.

7. The implantable device of claim 6, and further comprising:
a fourth buffer; and
wherein the controller is configured to copy waveform data from the first buffer to the fourth buffer upon receiving a signal from an external device.

8. The implantable device of claim 7, wherein the first, second, third, and fourth buffers are implemented in a single SRAM device.

9. The implantable device of claim 8, wherein the SRAM device is a 1 MB device that is arranged in a 512K by 16-bit manner.

10. The implantable device of claim 7, wherein the first, second, third, and fourth buffers are FIFO buffers.

11. The implantable device of claim 1, and further comprising:
a third buffer; and
wherein the controller is configured to copy waveform data from the first buffer to the third buffer and designate the copied waveform data as protected, thereby preventing the copied waveform data from being overwritten in the third buffer.

12. The implantable device of claim 11, wherein the controller automatically designates the copied waveform data as protected if at least one predetermined condition is satisfied.

13. The implantable device of claim 12, wherein the at least one predetermined condition comprises the patient's heart rate exceeding a threshold value.

14. The implantable device of claim 13, wherein the threshold value is about 220 beats per minute.

15. The implantable device of claim 12, wherein the implantable device is configured to determine a time interval between each adjacent pair of R-waves in the waveform data, and wherein the at least one predetermined condition comprises a threshold number of the intervals within a set of consecutive intervals exceeding a first upper limit, and at least one of the intervals within the set exceeding a second upper limit.

16. The implantable device of claim 15, wherein the threshold number of intervals is 6 intervals, and wherein the set of consecutive R-R intervals includes 8 consecutive intervals.

17. The implantable device of claim 1, wherein the regular intervals are about every 4 hours.

18. The implantable device of claim 17, wherein the controller is configured to copy about 10 seconds of waveform data from the first buffer to the second buffer every 4 hours.

19. The implantable device of claim 1, wherein the monitoring system is configured to monitor ECG signals.

20. A method of storing data in an implantable device to be implanted into a body of a patient, the implantable device configured to monitor biological signals of the patient, the method comprising:
continually storing waveform data corresponding to the monitored biological signals in a first buffer of the implantable device;
providing a plurality of event buffers in the implantable device; and
automatically copying waveform data from the first buffer to a first one of the event buffers at regular intervals.

21. The method of claim 20, and further comprising:
transmitting data from the first event buffer to an external device.

22. The method of claim 21, and further comprising:
transmitting data from the first event buffer to the external device at regular intervals.

23. The method of claim 21, and further comprising:
transmitting data from the first event buffer to the external device at least once per day.

24. The method of claim 20, wherein the implantable device is configured to communicate with the external device via wireless radio-frequency communications.

25. The method of claim 20, and further comprising:
detecting an event with the implantable device; and
automatically copying waveform data from the first buffer to a second one of the event buffers upon detection of the event.

26. The method of claim 25, and further comprising:
receiving a signal from an external device with the implantable device; and
automatically copying waveform data from the first buffer to a third one of the event buffers upon receiving the signal from the external device.

27. The method of claim 26, wherein the first buffer, and the first, second, and third event buffers, are implemented in a single SRAM device.

28. The method of claim 27, wherein the SRAM device is a 1 MB device that is arranged in a 512K by 16-bit manner.

29. The method of claim 26, wherein the first buffer, and the first, second, and third event buffers are FIFO buffers.

30. The method of claim 20, and further comprising:
copying waveform data from the first buffer to a second one of the event buffers; and
designating the waveform data copied to the second event buffer as protected, thereby preventing the copied waveform data from being overwritten in the second event buffer.

31. The method of claim 30, wherein the waveform data copied to the second event buffer is automatically designated as protected if at least one predetermined condition is satisfied.

32. The method of claim 31, wherein the at least one predetermined condition comprises the patient's heart rate exceeding a threshold value.

33. The method of claim 32, wherein the threshold value is about 220 beats per minute.

34. The method of claim 31, wherein the implantable device is configured to determine a time interval between each adjacent pair of R-waves in the waveform data, and wherein the at least one predetermined condition comprises a threshold number of the intervals within a set of consecutive intervals exceeding a first upper limit, and at least one of the intervals within the set exceeding a second upper limit.

35. The method of claim 34, wherein the threshold number of intervals is 6 intervals, and wherein the set of consecutive intervals includes 8 consecutive intervals.

36. The method of claim 20, wherein the regular intervals are about every 4 hours.

37. The method of claim 36, wherein about 10 seconds of waveform data are copied from the first buffer to the first event buffer about every 4 hours.

38. The method of claim 20, wherein the implantable device is configured to monitor ECG signals.

39. A system for monitoring a patient, comprising:
an implantable device to be implanted into a body of a patient, the implantable device configured to monitor biological signals of the patient, continually store waveform data corresponding to the monitored biological signals in a first buffer, and copy waveform data from the first buffer to a second buffer at regular intervals;
an external device configured to communicate with the implantable device; and
wherein the implantable device is configured to automatically transmit data from the second buffer to the external device.

40. An implantable device to be implanted into a body of a patient, the implantable device comprising:
a monitoring system configured to monitor biological signals of the patient, and generate corresponding waveform data;
a buffer for storing a portion of the waveform data upon an event being detected; and
a controller configured to automatically designate the stored waveform data as protected if the patient's heart rate is outside of at least one predetermined limit, thereby preventing the stored waveform data from being overwritten in the buffer.

41. A method of storing data in an implantable device to be implanted into a body of a patient, the implantable device configured to monitor biological signals of the patient and generate corresponding waveform data, the method comprising:
providing a buffer in the implantable device;
storing a portion of the waveform data in the buffer upon an event being detected; and
automatically designating the stored waveform data as protected if the patient's heart rate is outside of at least one predetermined limit, thereby preventing the stored waveform data from being overwritten in the buffer.
